(19) United States
(12) Patent Application Publication
(10) Pub. No.: US 2006/0223845 A1
(43) Pub. Date: Oct. 5, 2006

(54) Clopidogrel base suitable for pharmaceutical formulation and preparation thereof

(76) Inventors: Eran Turgeman, Herzelia (IL); Omer Malachi, Rehovot (IL)

Correspondence Address: KENYON & KENYON LLP, ONE BROADWAY, NEW YORK, NY, 10004, US

(21) Appl. No.: 11/362,330

(22) Filed: Feb. 24, 2006

Related U.S. Application Data
(60) Provisional application No. 60/656,738, filed on Feb. 24, 2005.
(60) Provisional application No. 60/659,544, filed on Mar. 7, 2005.
(60) Provisional application No. 60/661,701, filed on Mar. 14, 2005.
(60) Provisional application No. 60/675,371, filed on Apr. 26, 2005.

(51) Int. Cl. A61K 31/4743; C07D 498/02

(52) U.S. Cl. 514/301; 546/114

(57) ABSTRACT

Provided is clopidogrel base suitable for pharmaceutical formulation, and processes for its preparation.

### RELATED APPLICATION

This application claims the benefit of U.S. provisional application Nos. 60/656,738, filed Feb. 24, 2005; 60/659,544, filed Mar. 7, 2005; 60/661,701, filed Mar. 14, 2005 and 60/675,371, filed Apr. 26, 2005; herein incorporated by reference.

### FIELD OF THE INVENTION

The present invention relates to clopidogrel base suitable for pharmaceutical use.

### BACKGROUND OF THE INVENTION

Atherosclerosis is the buildup of plaque in the wall of the arteries leading to a thickening and a reduction in elasticity of the arteries. Atherosclerosis results from injury to the inside layer of the artery. The injury is caused by common activities and diseases such as high cholesterol, high blood pressure, smoking and infection.

Plaques form on the inner walls of the artery at these sites of injury. The plaques are mainly composed of fatty tissue and smooth muscle cells. The formation of plaque often leads to blood clotting due to platelet aggregation at the site of the injury. This clotting may result in a reduction or elimination of blood flow to vital organs, causing heart attacks or other serious conditions. The plaque may also rupture and send a blood clot through the artery, referred to as an embolus, which if deposited in a smaller blood vessel may completely block blood flow.

Antiplatelet activity is desirable in fighting the often fatal results of atherosclerosis. Clopidogrel is an inhibitor of induced platelet aggregation which acts by inhibiting the binding of adenosine diphosphate to its receptor. Clopidogrel is metabolized by the liver into active form. Its antiplatelet activity is extended in that it stops any platelet activity even up to ten days after administration.

The chemical name of clopidogrel is methyl (+)-(S)-α-(o-chlorophenyl)-6,7-dihydrothieno[3,2-c]pyridine-5(4H)-acetate. It has the following structure: 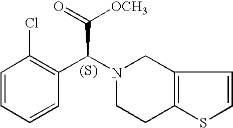 Clopidogrel is disclosed in U.S. Pat. No. 4,529,596 (EP 99802, JP 59027895), 6,258,961, 5,036,156 (EP 420706, JP 3120286), U.S. Pat. No. 6,080,875 (EP 971915, JP 2001513806) and U.S. Pat. No. 6,180,793 (EP 981529, JP 2001525829).

Clopidogrel's platelet inhibiting activity makes it an effective drug for reducing the incidence of ischemic strokes, heart attacks or claudication due to vascular diseases such as atherosclerosis. By inhibiting platelet aggregation, clopidogrel reduces the chance of arterial blockage, thus preventing strokes and heart attacks. U.S. Pat. No. 5,576,328 describes a method of preventing the occurrence of a secondary ischemic event by administration of clopidogrel, and is incorporated herein by reference.

Clopidogrel is presently administered as its bisulfate (syn. hydrogensulfate) salt. Clopidogrel bisulfate has an empirical formula of C₁₆H₁₆Cl NO₂S.H₂SO₄. It is currently being marketed as PLAVIX® tablets, which contain about 98 mg clopidogrel bisulfate (75 mg Clopidogrel base equivalent).

As evident by PLAVIX®, Clopidogrel is administered as a pharmaceutically acceptable salt to a patient. Clopidogrel base has been avoided for formulation inter alia because it exists as an oil that is highly contaminated with solvents and clopidogrel acid. An early patent on clopidogrel, U.S. Pat. No. 4,847,265, discloses that clopidogrel base “is an oil whereas its hydrochloride exists as a white powder. The oily products are usually difficult to purify and it is preferable to use for the preparation of pharmaceutical compositions crystalline products which can usually be purified by recrystallization.” A recently filed patent application (WO02/059128) also states: “As ‘Clopidogrel base’ is an oily liquid, in order to prepare a convenient formulation, the base is converted into a pharmaceutically acceptable salt.”

The existence of clopidogrel base as an oil makes formulation of clopidogrel base impractical since the oil contains unacceptable levels of solvents and clopidogrel acid. The Food and Drug Administration mandates for example presence of ethanol in an active pharmaceutical ingredient in a quantity less than 5000 ppm. There is a need in the art for clopidogrel base with such purity to meet the requirements of the Food and Drug Administration and GMP for use in preparation of a pharmaceutical formulation.

### SUMMARY OF THE INVENTION

In one embodiment, the present invention provides clopidogrel base having less than about 2% total residual organic solvent by weight. In other embodiments, it is less than about 1% by weight, less than about 0.5% by weight or less than about 1000 ppm total residual organic solvent. In one embodiment, the solvent is at least one of methanol, ethanol, or ethyl acetate.

In another embodiment, the present invention provides clopidogrel base having less than about 0.5% total impurities as area percentage HPLC. In other embodiments, it is less than about 0.3% or less than about 0.1% clopidogrel acid or less than about 0.02% clopidogrel acid as area percentage HPLC.

Also included are pharmaceutical compositions of clopidogrel base and methods of their use inhibiting platelet aggregation in a mammal.

In another embodiment, the present invention provides a process for preparing the clopidogrel base of any one of claim 1 to 9, comprising the steps of:
a) providing an oil comprising clopidogrel base and residual amount of at least one organic solvent; and
b) drying the oil in a Wiped Film Evaporator under reduced pressure.

### DETAILED DESCRIPTION OF THE INVENTION

The present invention provides processes for preparing clopidogrel base substantially free of solvents. This process allows for use of the base in pharmaceutical formulations on an industrial scale.

The term “industrial scale” refers to a batch size of at least about 0.2 kg, more preferably at least about 0.5 kg, and most preferably at least about 1.0 kg.

The Clopidogrel base of the invention is substantially free of solvent, preferably containing less than about 2% total solvent by weight, more preferably less than about 1% total solvent by weight, even more preferably less than about 0.5% total solvent by weight, and most preferably less than about 1000 ppm of total solvent. In one embodiment, the solvent is at least one of methanol, ethanol, ethyl acetate, or dichloromethane.

The Clopidogrel base of the present invention may be prepared by a Wiped Film Evaporator (WFE). A Wiped Film Evaporator is a device where clopidogrel base is wiped against a surface in the presence of reduced pressure, i.e., a pressure below one atmosphere. Typically, an internally revolving rotor equipped with either wipers, blades or similar device provides internal distribution and rapid transport of the clopidogrel base film. The vapors are removed via an outlet and separated from the clopidogrel base.

According to Gooch Thermal Systems (Lebanon, N.J.), the type of wiper or rotor design is a function of product behavior and process requirements, for example fouling/deposit formation tendencies, viscosity, residual moisture requirements, etc. Three basic types of rotors are typically used: Rigid blade rotor (fixed clearance between blade tip and heating surface), rotor with radially moving wipers (wiped film with either PTFE or graphite elements), and rotor with hinged free-swinging wiper blades (wiped film metal wipers or metal wipers with PTFE tips). One Wiped Film Evaporator that may be used is that available from POPE (Saukville, Wis.).

The WFE may be used with a jacket temperature of preferably about 20° C. to about 250° C., more preferably about 30° C. to about 200° C., and even more preferably about 50° C. to about 100° C. The feed rate is preferably about 0.1 ml/min to about 200 ml/min, more preferably about 0.1 ml/min to about 100 ml/min, and most preferably about 0.1 ml/min to about 50 ml/min. The tip speed is preferably about 0.1 μm/s to about 2 m/s, and more preferably about 1.57 m/s. The pressure is generally less than 1 atmosphere, preferably less than about 200 mm Hg, and more preferably less than about 100 mm Hg. Different parameters may be used for other types of Wiped Film Evaporators.

The solution of clopidogrel base used for feeding into the WFE may be prepared by routine methods known in the art. The starting material for the solution may be any salt of clopidogrel, such as the bisulfate salt or the camphor sulfonate salt. Alternatively, clopidogrel base may be purchased commercially in the form of an oil. One advantage of using the camphor sulfonate salt is that the camphor sulfonate is used for enantiomeric purification of clopidogrel, and thus the process of the present invention may be integrated with the enantiomer purification process as a subsequent step.

In one embodiment, clopidogrel camphor sulfonate is mixed with organic solvents such as, for example, at least one of C₁-C₅ chlorinated hydrocarbons, preferably C₁-C₃ chlorinated hydrocarbons, more preferably dichloromethane; cyclic or acyclic C₆ to C₈ alkanes, preferably hexane, cyclohexane, heptane, or cycloheptane; C₂-C₈ ethers, preferably C₄-C₆ ethers, more preferably methyl t-butyl ether (MTBE), diethyl ether, or tetrahydrofuran; C₄-C₇ ketones, preferably methyl ethyl ketone (MEK); C₆-C₉ aromatic hydrocarbons, preferably benzene or toluene; or C₃-C₇ esters, preferably ethyl acetate, propyl acetate, butyl acetatel, isobutyl acetate and isopropyl acetate. Most preferably, the organic solvent is ethyl acetate or dichloromethane.

An aqueous base is then added to free the clopidogrel base, which results in an aqueous phase and an organic phase. The Clopidogrel base moves to the organic phase, which is then separated from the aqueous phase. Separation may be by liquid phase separation or by solid liquid separation. Preferably, the base is an inorganic base, such as, for example, alkali metal and alkaline earth metal bases, particularly hydroxides, carbonates and bicarbonates, such as NaOH, BaOH₂, KOH and NaHCO₃ and mixtures thereof. The base may also be at least one of a tertiary amine, such as 1,8-diazabicyclo[5.4.0]undec-7-ene (DBU) or tributyl amine. Thus, the base may be anhydrous or in aqueous solution. Most preferably, the base is a mixture of NaOH and NaHCO₃.

The organic phase may then be evaporated to obtain clopidogrel base substantially free of solvent(s). For example, clopidogrel base in a solvent phase may be placed in a Wiped Film Evaporator to remove the solvent down to acceptable levels of residual solvent. Example 3 illustrates removal of ethyl acetate by WFE from the ethyl acetate phase without the steps of first evaporating ethyl acetate and dissolving the residue in another solvent.

Clopidogrel base may be prepared similarly from other organic phases containing other solvents.

Alternatively, before being placed in a WFE, the organic phase may be evaporated, preferably under reduced pressure, and dissolved in a volatile solvent. The volatile solvent is preferably one that has an azeotrope with the first solvent (such as ethyl acetate) used to prepare the clopidogrel base. Preferably, the volatile solvent is at least one C₁ to C₄ alcohol, more preferably at least one of methanol or ethanol, and most preferably methanol. The resulting solution is then fed to a Wiped Film Evaporator to produce clopidogrel base with acceptable amounts of residual solvent. Examples 1 and 2 illustrate a process of the invention, where the organic phase is first evaporated and then dissolved in methanol prior to being placed in the WFE.

Processes or apparatuses that may be used in addition to the Wiped Film Evaporator include, for example, spray drying (atomizing into heated air, such as nitrogen or argon, at above about 30° C.) and injection into a vacuum at a pressure below about 200 mm Hg, more preferably below about 100 mm Hg, flash evaporators, thin film evaporator, falling film stills, or rotary evaporators.

The clopidogrel base of the present invention is also substantially free of chemical impurities. The clopidogrel base of the present invention contains less than about 0.5% total impurities, as measured by HPLC. Specifically, the clopidogrel base of the present invention contains less than about 0.3%, more preferably less than about 0.1, and most preferably less than about 0.05% clopidogrel acid as area percentage HPLC. In one embodiment, the clopidogrel acid is 0.02% by HPLC. Clopidogrel acid has the following structure:

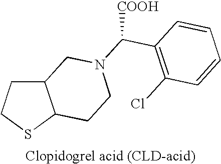

The present invention further provides pharmaceutical compositions comprising clopidogrel base and a pharmaceutically acceptable excipient.

In addition to the active ingredient(s), the pharmaceutical formulations of the present invention may contain one or more excipients. Excipients are added to the formulation for a variety of purposes. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid. Solid and liquid compositions may also be dyed using any pharmaceutically acceptable colorant to improve their appearance and/or facilitate patient identification of the product and unit dosage level.

In liquid pharmaceutical compositions of the present invention, clopidogrel base and any other solid excipients are dissolved or suspended in a liquid carrier such as water, vegetable oil, alcohol, polyethylene glycol, propylene glycol or glycerin.

Liquid compositions may also be dyed using any pharmaceutically acceptable colorant to improve their appearance and/or facilitate patient identification of the product and unit dosage level.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

The solid compositions of the present invention may include powders, granulates, aggregates and compacted compositions. The dosages include dosages suitable for oral, buccal, rectal, parenteral (including subcutaneous, intramuscular, and intravenous), inhalant and ophthalmic administration. The most suitable administration in any given case will depend on the nature and severity of the condition being treated. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs. The dosage form may be a capsule containing the composition, such as a powdered or granulated solid composition, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art. A composition for tableting or capsule filling may be prepared by wet granulation. In wet granulation, some or all of the active ingredients and excipients in powder form are blended and then further mixed in the presence of a liquid, typically water, which causes the powders to clump into granules. The granulate is screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting A capsule filling of the present invention may comprise any of the aforementioned blends and granulates that were described with reference to tableting, however, they are not subjected to a final tableting step.

Clopidogrel base is administered to a mammal, preferably a human in need thereof, to inhibit platelet aggregation and reduce the chance of a primary or secondary ischemic event such as a heart attack or stroke. In one embodiment, the clopidogrel base is administered as a gelcap.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way.

### EXAMPLES

Except for assays, or otherwise specified, percentages are by area percentage HPLC.

HPLC Method

Assays were carried out according to the U.S. Pharmacopoeia and performed by HPLC under the following parameters:

| | |
|---|---|
| Column: | XTerra phenyl 5 micron 4.6 × 250 mm |
| Eluent: | 500 ml aqueous solution of 5 g dodecyl sulfate |
| | sodium salt, pH adjusted to 3.0 by H₃PO₄, |
| | adding 420 ml acetonitrile and 80 ml methanol. |
| Flow rate: | 1.3 ml/min |
| Detector: | 220 nm |
| Sample volume: | 10 μL |
| Diluent: | Eluent. |

HS-GC method for measuring residual solvents

1) Chromatographic Conditions

| Column: | MXT-WAX (Crossbond Carbowax-PEG), 30 m × 0.53 mm ID, | |
|---|---|---|
| | 1.0 μm film thickness (Catalog No. 70655-Restek-USA) or equivalent. | |
| Carrier gas: | Helium, constant pressure, about 3.6 psi (5 ml/min. at 40° C.). | |
| Injection mode: | Headspace, split | |
| Split Ratio: | 1:4 by using HP-7694 headspace sampler (loop pressure technique) | |
| Detector: | Flame Ionization Detector. | |
| Make up gas: | Helium about 25 mL/min. | |
| Temperature: | Injector: | 180° C. |
| | Detector: | 250° C. |
| | Oven Program: | |
| | Initial temperature: | 40° C. |
| | Initial time: | 1.0 min. |

| | | | |
|---|---|---|---|
| Ramps. | Rate. | Final Temp. | Final Time. |
| 1 | 15.0° C./min. | 150° C. | 7.0 min. |

| | |
|---|---|
| Diluent: | N.N-Dimethylacetamide |

2) Headspace Conditions

| Apparatus: | HP-7694 headspace sampler (loop/pressure system) | |
|---|---|---|
| Vial pressure: | 12.5 psi | |
| Temperature: | Oven: | 90° C. |
| | Loop: | 100° C. |
| | Transfer line: | 110° C. |
| Times: | G.C. Cycle: | 24 min.* |
| | Sample eq.: | 35 min. |
| | Pressurize: | 0.20 min. |
| | Loop fill: | 0.10 min. |
| | Loop eq.: | 0.05 mn. |
| | Injection: | 0.50 min. |
| Shaking: | 1 (low) | |
| Loop volume: | 1 mL. | |
| Headspace vial: | 20 mL. | |

3) Standard Solution Preparation

3.1. Methanol Standard Preparation

The standard solution contain about 600 μg/mL Methanol.

3.2. Ethyl Acetate Standard Preparation

The standard solution contain about 1000 μg/mL Ethyl Acetate.

3.3. Ethanol Standard Preparation

The standard solution contain about 1000 μg/mL Ethanol.

3.4. Dichloromethane Standard Preparation

The standard solution contain about 120 μg/mL Dichloromethane.

4) Sample Analysis

About 100 mg of sample was dissolved in 0.5 mL of N,N-Dimethylacetamide.

5) Procedure

5.1. System Suitability Test
Standard Solutions are injected three times according to the headspace G.C. conditions and the following system suitability requirements should be met:
The RSD value for each individual triplicate response factors and for all six response factors should not be more than 10.0% for each residual solvent.
A resolution factor between any system peak or unidentified peak and the nearest analyte peak of not less than 1.0 should be achieved.

5.2. Calculations
Calculate the concentration in ppm of residual solvents in tested sample using the following formula:

$$ppm Residual Solvent = \frac{{r}_{Spl} \times {C}_{Std} \times 0.5}{{r}_{Std} \times {W}_{Spl}} = \frac{{r}_{Spl} \times 0.5}{R \cdot {F}_{Std} \times {W}_{Spl}}$$
r_{Spl} and r_{Std}: residual solvent peak area in sample solution chromatogram (r_{Spl}) and in standard solution chromatogram (r_{Std}) respectively.
C_{Std}: residual solvent concentration in injected standard solutions in μg/mL.
W_{Spl}: weight of sample in g.

$$R \cdot {F}_{Std} = \frac{{r}_{Std}}{{C}_{Std}}$$
:average standard response factor.

### Example 1

### Solvent Removal Using a Wiped Film Evaporator

Clopidogrel camphor sulfonate (120 grams) was dissolved in 360 ml of ethyl acetate in a stirred vessel. 240 ml of water and 16.3 g of 47% NaOH were added. 6.8 g of NaHCO₃ was gradually added, the content was mixed to dissolution and settled for phase separation. The upper organic phase was collected and evaporated in a rotavapor at a pressure of less than 100 mm Hg. The resulting oil was dissolved in methanol to give ca. 24% solution. The solution of clopidogrel base in methanol was evaporated in a Wiped Film Evaporator (WFE) (“POPE” 2 inch wipe film still). The jacket temperature was set to 60° C. The solution feed rate was about 200 ml/hr and the rotor speed was about 200 RPM. The product was collected as a thick paste at the bottom of the WFE and analyzed. The sample was found to be purely clopidogrel base.

R-Clopidogrel (CLD): 0.06%. Any unknown: <0.05%. CLD acid: <0.02%.

Residual solvents: Methanol: 4776 ppm. Ethyl acetate: 249 ppm.

Assay: 100.2%

### Example 2

### Solvent Removal Using a Wiped Film Evaporator

Clopidogrel camphor sulfonate (150 grams) was dissolved in 450 ml of dichloromethane. 300 ml of water and 20.4 g of 47% NaOH were added. 7.5 g of NaHCO₃ was gradually added, the content was mixed to dissolution and settled for phase separation. The lower organic phase was collected and evaporated in a vacuum evaporator. The resulting oil was dissolved in methanol to give ca. 20% solution. The solution of clopidogrel base in methanol was evaporated in a Wiped Film Evaporator (WFE) (“POPE” 2 inch wipe film still). The jacket temperature was set to 60° C. The solution feed rate was about 200 ml/hr and the rotor speed was about 200 RPM. The product was collected at the bottom of the WFE and analyzed. The sample was found to be purely clopidogrel base.

R-Clopidogrel (CLD): 0.04%. Any unknown: <0.52%. CLD Acid: 0.3%

Residual solvents: Methanol: 3071 ppm. Dichloromethane: 38 ppm.

Assay: 99.4%.

### Example 3

### Solvent Removal Using a Wiped Film Evaporator

Clopidogrel camphor sulfonate (100 grams) was dissolved in 200 ml of ethyl acetate in a stirred vessel. 200 ml of water and 5.6 g of 47% NaOH were added. 10.35 g of NaHCO₃ was gradually added, the contents mixed to dissolution, and settled for phase separation. The upper organic phase was collected and evaporated in a Wiped Film Evaporator (WFE) (“POPE” 2 inch wipe film still). The jacket temperature was set to 80° C. and the pressure was set to 60-65 mbar. The solution feed rate was about 350 ml/hr and the rotor speed was about 200 RPM. The product was collected as a thick paste at the bottom of the WFE and analyzed. The sample was found to be purely clopidogrel base.

Any unknown: <0.06%. CLD acid: <0.08%.

RRT, R-clopidogrel: 0.80: 0.13.

Residual solvents: Ethyl acetate: 2868 ppm.

Assay: 99.7%

### Example 4

### Solvent removal using a Rotary Evaporator

Clopidogrel camphor sulfonate (20 grams) was dissolved in 60 ml of Toluene in a stirred vessel. 40 ml of water and 2.7 g of 47% NaOH were added. 1.0 g of NaHCO3 was gradually added, the contents mixed to dissolution, and settled for phase separation. The upper organic phase was collected and evaporated in a Rotary Evaporator. The jacket temperature was set to 40° C. and the pressure was set to 10 mbar. The product was dissolved in 100 ml of Methanol and evaporated again in a Rotary Evaporator. The sample was found to be relativly dry clopidogrel base.

Residual solvents: Methanol: 6140 ppm.

What is claimed is:

1. Clopidogrel base having less than about 2% total residual organic solvent by weight.

2. The clopidogrel base of claim 1, wherein the total residual organic solvent is less than about 1% by weight.

3. The clopidogrel base of claim 1, wherein the total residual organic solvent is less than about 0.5% by weight.

4. The clopidogrel base of claim 1, having less than about 1000 ppm total residual organic solvent.

5. The Clopidogrel base of any one of claim 1 to 4, wherein the solvent is at least one of methanol, ethanol, or ethyl acetate.

6. Clopidogrel base having less than about 0.5% total impurities as area percentage HPLC.

7. Clopidogrel base having less than about 0.3% clopidogrel acid as area percentage HPLC.

8. Clopidogrel base of claim 7, having less than about 0.1% clopidogrel acid as area percentage HPLC.

9. Clopidogrel base of claim 8, having about 0.02% clopidogrel acid as area percentage HPLC.

10. A pharmaceutical composition comprising the clopidogrel base of any one of claim 1 to 9, and at least a pharmaceutically acceptable excipient.

11. A pharmaceutical composition comprising clopidogrel base and at least a pharmaceutically acceptable excipient.

12. A method of inhibiting platelet aggregation in a mammal comprising administering the pharmaceutical composition of claim 11 to the mammal.

13. A process for preparing the clopidogrel base of any one of claim 1 to 9, comprising the steps of:
c) providing an oil comprising clopidogrel base and residual amount of at least one organic solvent; and
d) drying the oil in a Wiped Film Evaporator under reduced pressure.

14. The process of claim 13, wherein the drying is carried out under the following conditions:
a) a jacket temperature of about 20° C. to about 250° C.;
b) a feed rate of about 0.1 ml/min to about 200 ml/min; and
c) a tip speed of about 0.1 m/s to about 2 m/s.

15. The process of claim 13, wherein the drying is carried out under the following conditions:
a) a jacket temperature of about 50° C. to about 100° C.;
b) a feed rate of about 0.1 ml/min to about 50 ml/min;
c) a tip speed of about 0.1 nm/s to about 2 m/s;
d) a pressure of less than about 100 mm Hg.

16. The process of claim 13, wherein the oil is prepared by a process comprising the steps of:
a) providing a salt of clopidogrel in an organic solvent;
b) reacting the salt with a base to obtain two phases, wherein Clopidogrel base moves into the organic phase;
c) separating the organic phase as the oil.

17. The process of claim 16, wherein the organic solvent is ethyl acetate.

18. The process of claim 13, wherein the oil is prepared by a process comprising the steps of:
a) providing a salt of clopidogrel in a first organic solvent;
b) reacting the salt with a base to obtain two phases, wherein Clopidogrel base moves into the organic phase;
c) separating the organic phase;
d) evaporating the first organic solvent from the organic phase;
e) adding a second organic solvent to obtain the oil, wherein the second organic solvent forms an azeotrope with the first organic solvent.

19. The process of claim 18, wherein the first organic solvent is ethyl acetate or dichloromethane, and the second organic solvent is methanol.
